(19) United States
(12) Patent Application Publication
(10) Pub. No.: US 2008/0177179 A1
(43) Pub. Date: Jul. 24, 2008

(54) Target Tissue Locator for Image Guided Radiotherapy

(75) Inventors: James Stubbs, Palo Alto, CA (US); Gregory K. Edmundson, Rough & Ready, CA (US); Srihari Yamanoor, Sunnyvale, CA (US); Russel M. Sampson, Palo Alto, CA (US)

Correspondence Address: CYTYC CORPORATION, 250 CAMPUS DRIVE, MARLBOROUGH, MA, 01752, US

(73) Assignee: Cytyc Corporation, Marlborough, MA (US)

(21) Appl. No.: 11/959,106

(22) Filed: Dec. 18, 2007

Related U.S. Application Data
(60) Provisional application No. 60/875,776, filed on Dec. 19, 2006.

(51) Int. Cl. A61B 5/00; A61B 18/18

(52) U.S. Cl. 600/431; 607/88

(57) ABSTRACT

The present invention relates to different methods of delineating target tissue from non-target tissue using differences in radiographic properties of a medical device.

### FIELD OF THE INVENTION

A method for treating tissue surrounding a cavity that is subject to a proliferative tissue disorder is provided. The method includes a tissue fixation device to position the tissue surrounding a resection cavity in a predetermined geometry. The tissue fixation device contains a negative contrast agent for localizing target tissue by visualizing the negative contrast agent in three dimensions. Methods of delineating target tissue from non-target tissue using differences in radiographic properties of a device are also presented.

### A BACKGROUND OF THE INVENTION

The invention relates generally to systems and methods for use in treating proliferative tissue disorders, and more particularly to systems and methods for the treatment of such disorders in the breast by positioning tissue and applying radiation.

Malignant tumors are often treated by surgical resection of the tumor to remove as much of the tumor as possible. Infiltration of the tumor cells into normal tissue surrounding the tumor, however, can limit the therapeutic value of surgical resection because the infiltration can be difficult or impossible to treat surgically. Radiation therapy can be used to supplement surgical resection by targeting the residual tumor margin after resection, with the goal of reducing its size or stabilizing it. Radiation therapy can be administered through one of several methods, or a combination of methods, including permanent or temporary interstitial brachytherapy, and external-beam radiation.

Brachytherapy refers to radiation therapy delivered by a spatially confined radioactive material inserted into the body at or near a tumor or other proliferative tissue disease site.

For example, brachytherapy is performed by implanting radiation sources directly into the tissue to be treated. Brachytherapy is most appropriate where 1) malignant tumor regrowth occurs locally, within 2 or 3 cm of the original boundary of the primary tumor site; 2) radiation therapy is a proven treatment for controlling the growth of the malignant tumor; and 3) there is a radiation dose-response relationship for the malignant tumor, but the dose that can be given safely with conventional external beam radiotherapy is limited by the tolerance of normal tissue. In brachytherapy, radiation doses are highest in close proximity to the radiotherapeutic source, providing a high tumor dose while sparing surrounding normal tissue. Interstitial brachytherapy is useful for treating malignant brain and breast tumors, among others.

Williams U.S. Pat. No. 5,429,582, entitled “Tumor Treatment,” describes a Brachytherapy method and apparatus for treating tissue surrounding a surgically excised tumor with radioactive emissions to kill any cancer cells that may be present in the tissue surrounding the excised tumor. In order to implement the radioactive emissions, Williams provides a catheter having an inflatable balloon at its distal end that defines a distensible reservoir. Following surgical removal of a tumor, the surgeon introduces the balloon catheter into the surgically created pocket left following removal of the tumor. The balloon is then inflated by injecting a fluid having one or more radionuclides into the distensible reservoir via a lumen in the catheter.

While brachytherapy procedures have successfully treated cancerous tissue, alternative radiation treatments are sometimes preferable, including radiation therapies which are delivered from a source external to the patient. For example, External Beam Radiation Therapy involves directing a “beam” of radiation from outside the patient's body, focused on the target tissue within a patient's body. The procedure is painless and often compared to the experience of having an x-ray.

As with any radiation therapy, the goal is to deliver a prescribed dose of radiation to the target tissue while minimizing damage to healthy tissue. More recent advances in radiation therapy such as Three-Dimensional Conformal Radiation Therapy (3DCRT) and Intensity Modulated Radiation Therapy (IMRT) have increased the precision of external radiation therapy with sophisticated shaping and directing of therapeutic radiation beams. In addition, imaging techniques allow delineation of a more complex planning target volume (“PTV”, PTV refers to the mass of tissue which includes both the residual malignancy as well as a margin of surrounding healthy tissue). These imaging procedures use cross-sectional imaging modalities including computed tomography (CT), magnetic resonance imaging (MRI), positron emission tomography (PET), single photon emission computed tomography (SPECT) and portal imaging to visualize target tissue. Treatment planning software combines the anatomical details from the imaging procedures and a PTV outlined by the physician, to optimize the number, size and shape of the radiotherapy beams used to treat the patient. The goal of the treatment plan is to deliver a conformal radiation dose to the PTV and minimize the radiation delivered to adjacent normal tissue outside the PTV.

In use, 3DCRT provides radiation beams shaped to “conform” to a target tissue volume, and with the ability to visualize and to arrange the radiation therapy beams, physicians can maximize coverage of the target tissue and minimize exposure to normal tissue. IMRT similarly conforms radiation beams to the size, shape and location of the target tissue by using hundreds to thousands of small, modulated radiation beams, striking the target tissue with varying intensities. The multitude of beams treats the target tissue and minimizes damage to healthy tissue. Yet, even the most advanced procedures require the patient and the target tissue to be properly positioned, and in some cases immobilized. Unfortunately, the irregular surface of a cavity created by the resection of tissue can make it difficult for the imaging techniques to determine the exact location of the target tissue, and even with the opportunity to completely map the target area, the unsupported tissue surrounding the resected cavity may shift during the procedure or between imaging and treatment, particularly where the treatment regimen involves radiation doses provided over the course of several days or weeks.

As a result, there is still a need for additional methods for delivering radiation from an external radiation source to tissue adjacent to a resected tissue cavity with a desired accuracy and without over-exposure of surrounding tissue. External beam radiation therapy involves directing or focusing a “beam” of radiation from the outside of a patient's body to an area of target tissue within the patient's body. The procedure is a non-invasive and relatively painless medical procedure which is used to treat abnormal or cancerous tissue in a patient. External radiation therapies rely on precise imaging and/or targeting techniques to locate tissues of interest for treatment. Patient positioning is often critical to the success of radiation therapy and great measures are often taken to ensure that patients are correctly positioned and immobilized. Even with the patient immobilized, internal movement of a patient's tissues as well as incorrect positioning of a patient's body can result in the damaging of normal healthy tissue by the radiation.

Radiographic imaging systems are commonly used in conjunction with external beam radiation systems (e.g., linear accelerators) to identify and target tissues. Targeting an external beam of radiation to a specific volume of interest requires a means of delineating the target tissues (e.g., a tumor), which have certain radiographic properties, from the surrounding non-target tissues (e.g., bone, soft tissue) which have different radiographic properties. There are different methods of delineating target tissue from non-target tissue using these differences in radiographic properties of the tissues. One such method is the insertion of radiographic markers around the targets volume's surface (or filing a cavity within the target) which further delineates the different radiographic properties of the tissues. The inserted markers are more radio-opaque than either the target tissue or the non-target tissue which allows the precise focusing of the external beam radiation to the target tissue. An example of such a method and markers can be found in copending, commonly assigned U.S. patent application Ser. No. 2005/0101860, filed Nov. 7, 2003, titled “Tissue Positioning Systems and Methods for Use with Radiation Therapy” which is incorporated by reference herein.

### SUMMARY OF THE INVENTION

The present invention provides methods, systems and devices for treating a proliferative tissue disorder by positioning tissue surrounding a resected tissue cavity and applying external radiation. The method includes first surgically resecting at least a portion of proliferative tissue and thereby creating a resection cavity. A tissue fixation device having an expandable surface is then provided, the expandable surface being sized and configured to reproducibly position tissue surrounding the resection cavity in a predetermined geometry upon expansion of the expandable surface into an expanded position. Next, the expandable-surface is positioned within the resection cavity and the expandable surface is expanded to position the tissue surrounding the resection cavity in the predetermined geometry. Finally, an external radiation treatment is applied to the tissue surrounding the resection cavity.

In another aspect of the invention, the resected cavity and the expanded tissue fixation device positioned therein can be visualized in three dimensions. The invention can also preferably include applying at least one of an external beam radiation treatment, a three-dimensional conformational radiation therapy treatment, and an intensity modulation radiation therapy treatment. The method may further include repeating the treatment steps several times during a treatment regimen.

In one embodiment, the expandable surface of the tissue fixation device includes a solid distensible surface defining a closed distensible chamber, and in a further embodiment the tissue fixation device is a balloon catheter. In yet a further embodiment, a second balloon can be positioned with in the first balloon. The balloons can be expanded with a variety of mediums including a non-radioactive substance. In other aspects of the invention, a treatment material is used to expand the balloon. The treatment material can include a drug such as a chemotherapy drug which is delivered through the wall of the balloon to the surrounding tissue.

In another aspect of the present invention, fiducial markers can be positioned on the tissue fixation device to determine the spatial location of the device and the surrounding PTV. For example, by determining the spatial position of the markers relative to the origin of a coordinate system of the treatment room (e.g., relative to the treatment beam isocenter or beam source), the location of the device and the PTV can be compared to their desired locations. If there are any changes in the PTV or in the location of the device, adjustments can be made to the position of the patient's body, the device, and/or the direction and/or shape of the planned radiation beams prior to initiation of the radiation fraction. The fiducial markers and their detection systems can be radio-opaque markers that are imaged radiographically or transponders that signal their position to a receiver system.

Another embodiment of the present invention includes a system for treating tissue surrounding a resected cavity that is subject to a proliferative tissue disorder. The system includes a tissue fixation device having a catheter body member with a proximal end, a distal end, an inner lumen, and an expandable surface element disposed proximate to the distal end of the body member, the expandable surface element being sized and configured to reproducibly position tissue surrounding a resected tissue cavity in a predetermined geometry upon expansion. An external radiation device is positioned outside the resected cavity such that the external radiation device can deliver a dose of radiation to the tissue surrounding the expandable surface element. With the tissue fixation device positioned within the resected tissue cavity and expanded to position the surrounding tissue, the accuracy of radiation from the external radiation device is greatly improved.

in yet a further embodiment, the invention includes a device for treating a proliferative tissue disorder after a lumpectomy procedure. The device including an elongate body member having an open proximal end defining a proximal port, a distal end and an inner lumen extending from the open proximal end, the elongate body member being sized for delivering an expandable surface element into a resection cavity created by a lumpectomy procedure. A spatial volume is defined by an expandable surface element disposed proximate to the distal end of the body member, the expandable surface element sized and configured to reproducibly position tissue surrounding a resected tissue cavity in a predetermined geometry upon expansion. The expandable surface element is size to fill a tissue cavity created in a breast during a lumpectomy procedure so as to position the surrounding tissue and allow an external radiation source to accurately deliver a dose of radiation. This invention generally relates to a method and device for the improved targeting of tissues during external beam radiation therapy (EBRT). Improved targeting of tissues during EBRT would allow for reduced volumes of tissue surrounding the target site that receives a therapeutic dose of radiation. Lower doses of radiation to non-target tissues would lower complications due to tissue toxicities as well as allowing for a reduction in the fractionation scheme.

The device of the present invention is comprised of a catheter with a proximal and distal end, connected to an expandable reservoir on the distal end such as a balloon.

In another aspect of the present invention, a device does not use a balloon catheter to target tissue. The device may be comprised of a catheter or introduction device for the placement of biocompatible materials, (foam, plastic, etc.) to occupy a resected tissue or natural cavity. The biocompatible material may or may not contain contrast medium.

The placement device serves as a guide for a suitably protected radiation source which is able to increase the radiation delivered to the target tissue from within the body. The placement device may also be used to guide other tools including those for treatment such as tools for the application of energy (e.g., heat, microwave, RF, etc.) to resect portions of tissue from the surrounding area.

In yet another aspect of the present invention, the biocompatible materials are bioabsorbable. Once the biocompatible materials are placed within the resected tissue or cavity, the material may remain within the patient's body for a period sufficient to complete the course of therapeutic treatment. Once the course of therapeutic treatment has been completed, the biocompatible material will be absorbed into the patient's body thus removing the requirement of additional invasive surgery or an additional visit to a doctor's office to undergo a procedure to remove a medical device.

### DETAILED DESCRIPTION OF THE INVENTION

The present invention provides systems and methods for treating proliferative tissue disorders, such as malignant tumors of the breast, by surgically resecting at least a portion of the proliferative tissue to create a resection cavity, followed by external radiation therapy of residual tumor margin. To improve the accuracy of the radiation treatment, a tissue fixation device is provided to position and/or stabilize the tissue surrounding the resected cavity.

External radiation therapies rely on precise imaging and/or targeting techniques, and any movement of the target tissue can introduce error. Patient positioning is often critical and great measures are taken to position and immobilize patients, including for example, marking the patient's skill and using foam body casts. Yet even with the patient immobilized, shifting of the target tissue still presents a problem, including for example, shifting of tissue as a result of the patient breathing and inconsistencies in the positioning of the patient's body between radiotherapy fractions.

Tissue cavities present an even greater difficulty because the tissue surrounding the cavity is often soft, irregular tissue which lacks the support usually provided by adjacent tissue. The irregular surface of the cavity wall, including the residual tumor margin, is therefore difficult to image. Unpredictable shifting of the tissue surrounding the cavity, possibly caused by slight patient movement, can further complicate the procedure and result in unacceptable movement of the target tissue. For example, where the target tissue changes position after visualization, but before radiation treatment, the shifting tissue may result in radiation beams encountering primarily healthy tissue. As a result, the residual tumor margin may be substantially untreated, while healthy tissue may be damaged by the treatment. The present invention overcomes these prior art problems by providing a tissue positioning device which can be inserted into the resected cavity and expanded to position the surrounding tissue in a predetermined geometry. The methods of the present invention also facilitate tissue imaging by positioning tissue against a defined surface.

The methods of the present invention also provide for systems and methods for the treatment of early stage breast cancers. For example, a breast cancer is removed surgically by resecting a lesion to create a resection cavity. After resection, the margin of the cavity is exposed to external beam radiation therapy. In order to improve the accuracy of the radiation treatment, a tissue fixation device is provided to position and/or stabilize the tissue surrounding the resected cavity. The method of the present invention provides for a means for directing or targeting the radiation beams using radiographic imaging of the device or other fiducial markers with real-time feedback for direction of the radiation beams.

The method of the present invention is based upon features of a balloon brachytherapy catheter (e.g., MammoSite System, Cytyc Corporation, Marlborough Mass.) as provided in U.S. Pat. Nos. 5,611,923 and 5,931,774, to Williams et al. and U.S. Pat. Nos. 6,200,257 and 6,413,204, and 6,482,142 to Winkler et al. all of which are incorporated by reference herein. Features of a balloon brachytherapy catheter which serves to bring radiation of the lumpectomy cavity margins can be applied in the method of the present invention for external beam radiation sources. Specifically, the implantation and inflation of a balloon catheter within a lumpectomy cavity provides for internal target fixation; external target fixation; and target localization.

The use of a balloon brachytherapy catheter allows for the fixation of an internal target.

The inflation of a balloon brachytherapy catheter in a resected lumpectomy cavity configures the target volume reproducibly in a shaped geometry (i.e., a spherical).

Having a regular and reproducible target volume allows for easier and more efficient radiation beam shaping to conform the radiation therapy to the target tissue, thus minimizing the radiation delivered to the adjacent healthy tissue. A more focused radiation beam also allows for reducing the size of the normal tissue margins typically added to the planning target volume (PTV).

The use of a balloon brachytherapy catheter also allows for the fixation of an external target. A portion of an implanted brachytherapy device will extend percutaneously through the skin. This external portion of the device can be coupled to a rigid fixture providing a means for holding-the target volume (i.e., the tissue surrounding the balloon) fixed relative to the 3-dimensional space of the treatment facilities (e.g., a radiation treatment couch). Thus, while the target volume is held in a constant position in regard to an external fixture, other patient tissue (e.g., breast tissue) remains slightly mobile relative to the same external fixture or even relative to the patient's body (e.g., chest wall). The fixation of the target tissue by an external means allows for better targeting of the radiation beams which again allows for reducing the size of the normal tissue margins typically added to the planning target volume. Also, the fixation of target tissue to an external fixture reduces the movement of target tissue due to movement of the patient. For instance, even the slightest movement of a patient can have a deleterious effect on locating and targeting tissues. In particular, the target motion of lumpectomy cavities due to a patient's respiration can affect beam efficiencies. Thus, the fixation of target tissue can reduce or eliminate the movement of target tissue by involuntary patient movements.

The use of a balloon brachytherapy catheter also allows for target localization. A brachytherapy balloon inflated with air or other contrast material provides a radiographic method for real time aiming of the planned radiation beams. The location of the inflated device can be otherwise ascertained via a number of other fiducial marking systems that can telegraph their location within the treatment room (specifically relative to the linear accelerator's isocenter). An example of this capability is target localization via the Beacon® Electromagnetic Transponder (Calypso Medical Seattle, Wash.). Thus, radiation beams can be shaped on the fly to account for target location changes or can provide a means to turn the beam on and off as the target moves in space and intersects the beams.

The present invention including a system for treating tissue surrounding a resected cavity that is subject to a proliferative tissue disorder. The system includes a tissue fixation device which includes a catheter body member having a proximal end, a distal end, an inner lumen and an expandable surface element. Expandable surface element is preferably disposed proximate to distal end of catheter body member and is sized and configured to reproducibly position tissue surrounding a resected tissue cavity in a predetermined geometry upon expansion. The system also includes an external radiation device positioned outside the resected cavity such that external radiation device can deliver a dose of radiation to the tissue surrounding expandable surface element. External radiation device can be any external radiation source known in the art or later developed, however, in preferred embodiments of the invention, precisely targeted sources such as those used in 3DCRT and IMT are employed. The tissue fixation device can be positioned within a resected tissue cavity, for example within a patient's breast following a lumpectomy, and expanded to position the surrounding tissue such that the dose of radiation beams from external radiation device is accurately delivered.

The expandable surface of the device can be defined by an inflatable balloon. It will be understood that the term “balloon” is intended to include distensible devices which can be, but need not be, constructed of an elastic material. The balloon of the present invention may include the variety of balloons or other distensible devices designed for use with surgical catheters. The balloon can be expanded by injecting an inflation material through body and into the balloon, and preferably, the inflation material comprises non-radioactive liquids or gases.

In one embodiment, the balloon is constructed of a solid material that is substantially impermeable to active components of a treatment fluid with which it can be filled, and is also impermeable to body fluids, e.g., blood, cerebrospinal fluid, and the like. An impermeable balloon is useful in conjunction with a treatment fluid, to prevent the material from escaping the treatment device and contaminating the surgical field or tissues of the patient.

In another embodiment, the balloon is permeable to a treatment fluid, and permits a treatment fluid to pass out of device and into a body lumen or cavity. A permeable balloon is useful when the treatment fluid is a drug such as for example, a chemotherapeutic agent which must contact tissue to be effective. U.S. Pat. Nos. 5,611,923 and 5,931,774 to Williams et al. disclose exemplary permeable balloons and treatment substances. Semi-permeable balloons can also find use in the method of the present invention. For example, a semi-permeable material that is capable of preventing the passage of a material through the balloon wall can be used to contain a treatment fluid, where certain fluid components can pass through the membrane while the components of the treatment fluid are retained within the balloon. Examples of which can be found in co-pending, commonly assigned U.S. patent application Ser. No. 2005-0107653.

In another embodiment, materials may be impregnated or incorporated into the expandable surface of the implantable device. For example, the expandable surface may be made of metal, be coated with a metal, or may contain metal in a matrix which is integrated into the expandable surface of the device. When the expandable surface is deployed in a patient's body, the metal in the expandable surface provides contrast between soft tissue and the surface of the device and thus imaging capability of the device becomes integral to the device (i.e., no longer needs a contrast agent). Examples of metals which may be incorporated into the expandable surface include any high Z material such as gold, silver, tungsten, etc., or stretchable metallized fabric mesh which is preferably knitted from a nylon and spandex knit plated with gold or other conductive material.

Although the balloon and body member can mate in a variety of ways, in some embodiments, the balloon is mated to body member at substantially a single point on, or a single side of, the balloon body. Such attachment permits the balloon (e.g., a spherical balloon) to maintain a substantially constant (e.g., spherical) shape over a range of inflation volumes. That is, the balloon is not constrained in shape by multiple attachment points to the body member, as is commonly the case with, e.g., balloons for Foley catheters. In other embodiments, the balloon is attached to the body member at multiple points on the balloon body, while allowing the balloon to maintain a constant shape over a range of inflation sizes. For example, a balloon attached to a body member at both distal and proximal points on the balloon body can be unconstrained upon inflation where the body member includes an expansion element (e.g., a slidable engagement element) that permits the body member to adjust in length as the balloon expands or contracts. A balloon which maintains a substantially constant shape over a range of inflation volumes permits a surgeon to select a balloon with less concern over the size of the cavity.

The body member of device provides a means for positioning expandable surface within the resected tissue cavity and provides a path for delivering inflation material (if used).

Although the exemplary body members have a tubular construction, one of skill in the art will appreciate that body members can have a variety of shapes and sizes. Body members suitable for use in the invention can include catheters which are known in the art. Although body members can be constructed of a variety of materials, in one embodiment the body member material is silicone, preferably a silicone that is at least partially radio-opaque, thus facilitating x-ray location of body member after insertion of device. The body members can also include conventional adapters for attachment to a treatment fluid receptacle and the balloon, as well as devices, e.g., right-angle devices, for conforming body members to contours of the patient's body.

The position of the device with in a patient's body can also be determined using fiducial markers. By positioning the markers on the device (for example on expandable surface member or on body member), a user can determine the spatial position of the device and the surrounding target tissue. The spatial data can be used to correct errors in target tissue location by adjusting the patient's body location on the treatment couch or by altering the radiotherapy beams' shape and direction. Fiducial markers are discussed in more detail below.

The device of the present invention can also include a variety of alternative embodiments designed to facilitate tissue positioning. For example, the device can include multiple spatial volumes, as well as, a variety of shapes adapted to conform and shape the resected cavity. In addition, the expandable surface can be positioned on and mated with tubular body member in various ways to facilitate placement of the expandable surface within a tissue cavity. The expandable surface can also be adapted to allow delivery of a treatment material to the tissue surrounding the cavity.

The invention also contemplates the use of multiple balloons, e.g., a double-walled structure. Such a balloon can comprise, for example, an impermeable inner wall and a permeable outer wall. In this embodiment, the inner balloon can be filled with, e.g., a radioactive treatment fluid, while the outer balloon (i.e., the space between the inner and outer balloon walls) is filled with a chemotherapeutic treatment fluid. This embodiment allows multiple modes of therapy (e.g., chemotherapy, brachytherapy and external radiation) to be administered with a single device. In this double-walled balloon embodiment the two balloons can be inflated with two treatment fluids at the same time or at different times during therapy. Inflation of an inner balloon can provide pressure on an outer balloon, which can cause the outer balloon to expand, or can force or urge fluid in the space between the inner and outer balloon walls through the membrane of a porous outer balloon. Higher-order balloons, e.g., triple-walled balloons, can also be used in the inventive devices.

The expandable surface can include a variety of shapes. For example, a generally spherical cavity can be filled and made to conform to a substantially spherical expandable surface, while it may be preferable to use an elongated expandable surface to position tissue surrounding an elongated body cavity. In some cases, it may be desirable to use an expandable surface which has a different shape than that of the resected cavity so that when expanded, the expandable surface applies increased relative pressure to part of the cavity wall, e.g. applies pressure to a problem area. One of skill in the art will appreciate that the inner and outer expandable surfaces may define a variety of shapes depending on the form of the original resected cavity and on the desired shape of the cavity after conforming to the expandable surface, including by way of non-limiting example, a cube, a parallelepiped, a cylinder, a tetrahedron, a prism, an irregular shape or combinations thereof.

In yet a further embodiment of the device having an expandable surface which resides within inner lumen of tubular body. In this embodiment, the inner lumen extends the length of body and expandable surface is fixedly attached at distal end body. As an inflation material is injected through inner lumen, expandable surface expands outwardly from tubular body. This device may be particularly advantageous for positioning tissue surrounding a spherical tissue cavity because the expandable surface can hold a generally spherical shape over a range of volumes. It may be desirable when body member of device is positioned proximate to a body cavity prior to expanding.

In some embodiments, the inventive devices are provided in pre-assembled form, i.e., the components are assembled in advance of a surgical insertion procedure. In certain embodiments, however, the inventive devices are configured to permit modular assembly of components, e.g., by a surgeon. Thus, for example, a treatment fluid receptacle can be provided with an element adapted for connection to any one of a plurality of catheters.

The connection element can be, e.g., any element known in the art for effecting connection between components such as catheters, injection ports, and the like.

Illustrative connectors include luer adapters and the like. In this embodiment, a variety of catheters and balloons can be provided, each of which is adapted for facile connection to the treatment fluid receptacle. The surgeon can then select an appropriate size and shape of expandable surface (e.g. balloon) for treatment of a particular proliferative disorder without need for providing several treatment fluid receptacles. The catheter and balloon can be selected according to the results of pre-operative tests (e.g., x-ray, MRI, and the like), or the selection can be made based on observation, during a surgical procedure, of the target cavity (e.g., a surgical cavity resulting from tumor excision). When the surgeon selects an appropriate balloon (e.g., a balloon having a size and shape suitable for placement in a body cavity), the catheter and balloon can then be attached to the pre-selected treatment fluid receptacle, thereby assembling the treatment device.

A method of the present invention can be used to treat a variety of proliferative tissue disorders including malignant breast and brain tumors. Many breast cancer patients are candidates for breast conservation surgery, also known as lumpectomy, a procedure that is generally performed on early stage, smaller tumors. Breast conservation surgery may be followed by radiation therapy to reduce the chance of recurrences near the original tumor site. Providing a strong direct dose to the effected area can destroy remaining cancer cells and help prevent such recurrences.

Surgery and radiation therapy are also the standard treatments for malignancies which develop in other areas of the body such as brain tumors. The goal of surgery is to remove as much of the tumor as possible without damaging vital brain tissue. The ability to remove the entire malignant tumor is limited by its tendency to infiltrate adjacent normal tissue. Partial removal reduces the amount of tumor to be treated by radiation therapy and, under some circumstances, helps to relieve symptoms by reducing pressure on the brain.

A method according to the invention for treating these and other malignancies begins by surgical resection of a tumor site to remove at least a portion of the cancerous tumor and create a resection cavity. Following tumor resection, device is placed into the tumor resection cavity. This can occur prior to closing the surgical site such that the surgeon intra-operatively places the device, or alternatively device can be inserted once the patient has sufficiently recovered from the surgery. In the later case, a new incision for introduction of device can be created. In either case, expandable surface, which is preferably sized and configured to reproducibly position tissue surrounding the resection cavity in a predetermined geometry, is then expanded within the resected tissue cavity.

Where expandable surface is defined by a balloon, the balloon can be expanded by delivering an inflation material through the inner lumen into the balloon to expand the balloon.

Expandable surface can be selected such that, upon expansion, expandable surface compresses the tissue which is being treated, or the surrounding tissues. Thus, where expandable surface is a balloon, it can be selected to have a desired size, and the amount of injected material can be adjusted to inflate the balloon to the desired size. When inflated expandable surface preferable fills a volume of at least about 4 cm³, and even more preferably it is capable of filling a volume of at least about 35 cm³. Preferable inflation volumes range from 35 cm³ to 150 cm³. In general, when deflated the balloon should have a small profile, e.g., a small size to permit facile placement in and removal from the patient's body and to minimize the size of a surgical incision needed to place and remove the balloon at the desired site of action.

With device expanded, it supports the tissue surrounding the tissue cavity and reduces tissue shifting. In addition, expandable surface can position the tissue in a predetermined geometry. For example, a spherical expandable surface can position the tissue surrounding the tissue cavity in a generally spherical shape. With the tissue positioned, a defined surface is provides so that radiation can more accurately be delivered to the previously irregular tissue cavity walls. In addition, device helps reduce error in the treatment procedure introduced by tissue movement. The positioning and stabilization provided by device greatly improves the effectiveness of radiation therapy by facilitating radiation dosing and improving its accuracy. The result is a treatment method which concentrates radiation on target tissue and helps to preserve the surrounding healthy tissue.

Prior to delivering radiation, but after expanding the expandable surface, device and the surrounding tissue can preferably be visualized with an imaging device, including by way of non-limiting example, x-ray, MRI, CT scan, PET, SPECT and combinations thereof.

These imaging devices provide a picture of the device and the surrounding tissue to assist with the planning of external radiation therapy. To aid with visualization, device can be constructed of materials which highlight expandable surface during the imaging procedure, for example, the expandable surface may be constructed of a radio opaque material. Alternatively, radiation transparent materials can be used so that tissue imaging is not blocked by the expandable surface. In either embodiment, the expandable surface can be inflated with a diagnostic imaging agent, including radioactive ray absorbent material, such as air, water or a contrast material.

In the case of external radiation therapies such as 3DCRT and IMRT, the imaging procedures provide a map of the residual tissue margin and assist with targeting tissue for radiation dosing. The radiation beams are then adapted for delivering a very precise radiation dose to the target tissue. With device 10 positioning the tissue surrounding the resection cavity, there is less danger of the target tissue shifting (within the body) and thus having the planned radiation missing the PTV and needlessly damaging healthy tissue.

Some treatment regimens require repeated radiation dosing over a course of days or weeks, and device can be used in those cases to repeatedly position the tissue surrounding the resected tissue cavity. For example, after delivering radiation from the external source, the expandable surface is collapsed. Although device can be removed after the step of collapsing, preferably the device is left within the tissue cavity between radiation treatments. When a subsequent radiation treatment is to be delivered, the expandable surface can be expanded and the adjacent tissue can be repositioned for another imaging step and/or radiation dose. These steps can be repeated as necessary over a course of a treatment regimen. Alternatively, the device is left within the tissue cavity and is maintained at a generally constant volume of expansion/inflation during an entire course of radiation therapy.

In another embodiment of the present invention, the target tissue is localized in 3-D space using imaging modalities (KV or MV photons) integrated onto the linear accelerator.

The localization of the target tissue takes advantage of an implantable targeting device that has sufficient contrast with soft tissue. The implantable device fixates the target tissues in 3-D space in a reproducible fashion by expansion of a portion of the device within the target tissue. The expandable portion of the device contains or is composed of a metallic coating or has a metal matrix integrated into the expandable surface. The radiation beam is aimed at the target tissue from multiple beam angles around the patient's body. The beam's profile in each beam angle is shaped to optimally conform to the outer surface (as seen in the beam's eye view) of the target volume.

The aforementioned method may be improved if the beam has its outer shape governed by manipulating the specific configuration of the leaves of a multi-leaf collimator. The multi-leaf collimator shapes the outer boundary of the beam to follow the shape of the tissue fixator by applying a margin (uniform or not) to the surface of the device (as seen in the beam's eye view). The margin can be of any desired size from a few millimeters to several centimeters. The outer shape can be set by using a permanently fixed shielding block that is affixed below the gantry of the linac head and shields out the portions of the beam outside the desired target volume. Dose sparing for non-target tissue can be accomplished by blocking a portion of the beam that is aimed within the fixator/locator device (central beam shielding). The central beam shielding can be accomplished using the multi-leaf collimator. The central beam shielding can be most easily accomplished using a fixed shield block that is affixed below the gantry of the linac head and shields out portions of the beam aimed entirely within the implanted fixator/locator device. The central shield work best as a cylinder composed of high Z material such as aluminum, lead, palladium, gold, titanium, molybdenum, niobium, tantalum, tungsten, and pewter or alloys containing high Z materials such as a pewtalloy or bend alloy.

Another embodiment of the invention incorporates fiducial markers that provide real-time, wireless information about the device's spatial position relative to the origin of a coordinate system in the treatment room (e.g., the isocenter of the radiation delivery device or the radiation beam's source location). The spatial position data can be used to correct errors in target volume location. For example, by adjusting the patient's body position on the treatment couch and/or altering the radiotherapy beams' shape and direction to correct for the altered PTV position. Preferably, the real-time, wireless feedback allows correction of positioning errors prior to delivery of each fraction of radiation. Fiduciary markers can also provide users more a more accurate PTV position and thereby allow greater normal tissue sparing and smaller normal tissue margins within the PTV. Preferably, the fiducial markers and their detection systems are radio-opaque markers that are imaged radiographically (e.g., fluoroscopically) or transponders that signal their positions to a receiver system. An exemplary fiducial marker is the Beacon Transponder, made by Calypso Medical Technologies of Seattle, Wash.

Positioning fiducial markers on device provides an advantage over other placements of such markers (e.g. placement within a tumor). For example, by placing a fiducial marker on expandable surface member, the position of the expandable surface can be precisely determined and the amount of expansion can be adjusted. In addition, a marker positioned on the outside of device can be used to delineate the surrounding target tissue (a.k.a. the PTV). As an additional benefit of having the marker positioned on the device, a separate insertion step is not required for the marker. Also, when the device is removed, the marker will also be removed, thereby assuring that foreign objects are not left permanently in the patient at the conclusion of the treatment.

In yet another embodiment of the present invention, fiducial markers may be comprised of surgical clips which may be used to facilitate the localization of a surgical cavity or surgical margin during a follow-up reexamination. The surgical clip is made of metal or other radio-opaque material that is attached or “stapled” onto the surgical margin through a variety of mechanical means. In particular, the surgical clips may be deployed within the patient's body from the expandable surface of an implantable device described supra.

Alternatively, the treatment material may be mated to the expandable surface such that after insertion of device, expandable surface delivers the treatment material to surrounding tissue. The treatment material can diffuse from expandable surface to tissue and/or the treatment material may be delivered as the expandable surface presses against the resected cavity walls and contacts tissue. In yet a further embodiment, the treatment material may be positioned on only part of the expandable surface. Regardless of the method of delivery, the treatment materials may include, by way of non-limiting example, a chemotherapy agent, an anti-neoplastic agent, an anti-angiogenesis agent, an immunomodulator, a hormonal agent, an immunotherapeutic agent, an antibiotic, a radio sensitizing agent, and combinations thereof.

A person of ordinary skill in the art will appreciate further features and advantages of the invention based on the above-described embodiments. Accordingly, the invention is not to be limited by what has been particularly shown and described, except as indicated by the appended claims. All publication and references cited herein are expressly incorporated herein by reference in their entity. The invention is described further in the following non-limiting examples.

The invention can be embodied in other specific forms Without departing from the spirit or essential characteristics thereof. The present embodiments are therefore to be considered in all respects as illustrative and not restrictive. The scope of the invention is indicated by the appended claims, rather than by the foregoing description, and all changes which come within the meaning and range of equivalency of the claims are therefore intended to be embraced therein.

1. A method for localizing target tissue in a patient, comprising: (a) surgically resecting at least a portion of the target tissue and thereby creating a resection cavity comprising a target volume; (b) applying an amount of a negative contrast agent to said resection cavity such that said target volume is filled with said a bioabsorbable negative contrast agent; and (c) localizing the target tissue by visualizing the negative contrast agent in three dimensions, and (d) applying the external radiation treatment to said target tissue.

2. The method of claim 1, wherein the step of surgically resecting is performed during a lumpectomy procedure.

3. The method of claim 1, wherein the external radiation treatment is an external beam radiation treatment, three-dimensional conformational radiation therapy treatment or intensity modulation radiation therapy treatment.

4. The method of claim 1 wherein said negative contrast agent has a density of less than 1.04 grams/cc.

5. The method of claim 1 wherein said negative contrast agent is a foam or gel.

6. The method of claim 1 wherein said negative contrast agent is bioabsorbable.

7. A method for localizing target tissue in a patient, comprising: (a) applying an amount of a negative contrast agent to a body cavity such that said cavity is filled with said negative contrast agent; (b) localizing a target tissue by visualizing the negative contrast agent in three dimensions wherein said target tissue is proximate to said cavity, and (d) applying the external radiation treatment to said target tissue.

8. The method of claim 7, wherein the external radiation treatment is an external beam radiation treatment, three-dimensional conformational radiation therapy treatment or intensity modulation radiation therapy treatment.

9. The method of claim 7 wherein said negative contrast agent has a density of less than 1.04 grams/cc.

10. The method of claim 7 wherein said negative contrast agent is a foam or gel.

11. The method of claim 7 wherein said negative contrast agent is bioabsorbable.

12. A method for localizing target tissue in a patient, comprising: (a) applying an amount of a negative contrast agent to a body cavity such that said cavity is filled with said negative contrast agent; (b) localizing the target tissue by visualizing the negative contrast agent in three dimensions, and (d) applying radiation treatment to said target tissue.

13. The method of claim 12 wherein said negative contrast agent has a density of less than 1.04 grams/cc.

14. The method of claim 12 wherein said negative contrast agent is a foam or gel.

15. The method of claim 12 wherein said negative contrast agent is bioabsorbable.

16. A method for treating a proliferative tissue disorder, comprising: (a) surgically resecting at least a portion of the proliferative tissue and thereby creating a resection cavity; (b) providing a tissue fixation device having an expandable surface sized and configured to reproducibly position tissue surrounding the resection cavity in a predetermined geometry upon expansion of the expandable surface into an expanded position and wherein said expandable surface is comprised of metal or a metal matrix; (c) positioning the tissue fixation device so that the expandable surface is within the resection cavity; (d)
expanding the expandable surface to position the tissue surrounding the resection cavity in the predetermined geometry; and (e) applying an external radiation treatment to the tissue surrounding the resection cavity.
